(19) United States
(12) Patent Application Publication
(10) Pub. No.: US 2016/0256406 A1
(43) Pub. Date: Sep. 8, 2016

(54) OLIGOMERIC/POLYMERIC SILICONE FLUIDS FOR USE IN TRANSDERMAL DRUG DELIVERY SYSTEMS

(71) Applicant: Noven Pharmaceuticals, Inc., Miami, FL (US)

(72) Inventors: Puchun Liu, Miami, FL (US); Steven Dinh, Miami, FL (US); Jun Liao, Miami, FL (US); Tsuyoshi Endo, Miami, FL (US)

(73) Assignee: Noven Pharmaceuticals, Inc., Miami, FL (US)

(21) Appl. No.: 15/054,730

(22) Filed: Feb. 26, 2016

Related U.S. Application Data
(60) Provisional application No. 62/128,197, filed on Mar. 4, 2015.

(51) Int. Cl. A61K 9/70; A61K 47/34; A61K 31/4458; A61K 31/137; A61K 31/325

(52) U.S. Cl. CPC A61K 9/7038; A61K 31/137; A61K 31/325; A61K 31/4458; A61K 47/34

(57) ABSTRACT

Described are oligomeric/polymeric silicone fluids useful in transdermal drug delivery systems. Also described are compositions for the transdermal delivery of a drug comprising a drug-containing polymer matrix comprising a drug; a carrier polymer, and an oligomeric/polymeric silicone fluid having repeating —Si(CH₃)₂—O— units. Related methods and uses also are described.

### CROSS-REFERENCE TO RELATED PATENT APPLICATIONS

This application claims the priority benefits under 35 USC §119(e) to U.S. provisional application 62/128,197, filed Mar. 4, 2015, the entire contents of which are incorporated herein by reference in its entirety.

### FIELD

Described here are oligomeric/polymeric silicone fluids useful in transdermal drug delivery systems, methods of making them, pressure-sensitive adhesive compositions and transdermal drug delivery systems comprising them, and methods of effecting transdermal drug delivery using them.

### BACKGROUND

Many factors influence the design and performance of transdermal drug delivery compositions. These include the individual drugs themselves, the physical and chemical characteristics of the compositions' components and their performance and behavior relative to other components, external and environmental conditions during manufacturing and storage, properties of the application site, the desired rate of drug delivery and therapeutic onset, the desired drug delivery profile, and the intended duration of delivery, among others.

Pressure-sensitive adhesive compositions for the transdermal delivery of drugs are known, but there remains a need for compositions that exhibit suitable physical and pharmacokinetic properties. For example, there remains a need for compositions that can achieve high drug-loading and desirable pharmacokinetic properties while retaining satisfactory physical properties and satisfactory wear properties. There also remains a need for transdermal drug delivery systems for primary and secondary amine drugs that exhibit desirable pharmacokinetic properties while retaining satisfactory physical properties and satisfactory wear properties.

### SUMMARY

In accordance with some embodiments, there are provided compositions for the transdermal delivery of a drug in the form of a flexible finite system for topical application, comprising a drug-containing polymer matrix comprising (i) a drug; (ii) a carrier polymer, and (iii) an oligomeric/polymeric silicone fluid having repeating —Si(CH₃)₂—O— units. In some embodiments, the polymer matrix comprises from 2.5 to 50% by weight of the oligomeric/polymeric silicone fluid, or from 7.5 to 20% by weight of the oligomeric/polymeric silicone fluid. In some embodiments, the oligomeric/polymeric silicone fluid comprises a linear oligomeric/polymeric silicone. In specific embodiments, the linear oligomeric/polymeric silicone is selected from the group consisting of hexamethyldisiloxane, octamethyltrisiloxane, decamethyltetrasiloxane, and dodecamethylpentasiloxane. In some embodiments, the oligomeric/polymeric silicone fluid comprises a cyclic oligomeric/polymeric silicone. In specific embodiments, the cyclic oligomeric/polymeric silicone fluid is selected from the group consisting of hexamethylcyclotrisiloxane, octamethylcyclotetrasiloxane, decamethylcyclopentaasiloxane, and dodecamethylcyclohexasiloxane.

In accordance with any of these embodiments, the polymer matrix may comprise an acrylic polymer, such as one or more selected from the group consisting of non-functional acrylic polymers, hydroxy-functional acrylic polymers, and carboxy-functional acrylic polymers.

In accordance with any of these embodiments, the drug may be selected from the group consisting of primary and secondary amine drugs in free base form, such as amphetamine, rivastigmine, methylphenidate and clonidine.

In accordance with any of these embodiments, the polymer matrix may be substantially free or free of silicone-containing pressure-sensitive adhesives.

In accordance with any of these embodiments, the composition may further comprise a backing layer and/or a release liner.

In accordance with other embodiments, there are provided methods for the transdermal delivery of a drug, comprising topically applying a composition as described herein to the skin or mucosa of a subject in need thereof

In accordance with other embodiments, there are provided compositions as described herein for use in transdermally delivering a drug, and uses of compositions described herein in the preparation of a medicament for transdermally delivering a drug.

In accordance with other embodiments, there are provided methods of preparing a composition for the transdermal delivery of a drug in the form of a flexible finite system for topical application, comprising preparing a drug-containing polymer matrix comprising (i) a drug; (ii) a carrier polymer, and an oligomeric/polymeric silicone fluid having repeating —Si(CH₃)₂—O— units. In some embodiments, the polymer matrix is prepared to comprise from 2.5 to 50% by weight of the oligomeric/polymeric silicone fluid.

### BRIEF DESCRIPTION OF THE DRAWINGS

FIGS. 1A-C show the in vitro flux (μg/cm²/hr) through human cadaver skin of amphetamine over 24 hours from a drug-containing polymer matrix as described herein comprising 20% wt cyclomethicone as compared to a comparison composition polymer matrix formulated without cyclomethicone.

FIGS. 2A-B show the in vitro flux (μg/cm²/hr) through human cadaver skin of rivastigmine over 24 hours from a drug-containing polymer matrix as described herein comprising 20% wt cyclomethicone as compared to a comparison composition polymer matrix formulated without cyclomethicone.

FIGS. 3A-C show the in vitro flux (μg/cm²/hr) through human cadaver skin of methylphenidate over 24 hours from a drug-containing polymer matrix as described herein comprising 20% wt cyclomethicone as compared to a comparison composition polymer matrix formulated without cyclomethicone.

### DETAILED DESCRIPTION

Described here are oligomeric/polymeric silicone fluids useful in transdermal drug delivery systems, methods of making them, pressure-sensitive adhesive compositions and transdermal drug delivery systems comprising them, and methods of effecting transdermal drug delivery using them. In some embodiments, the oligomeric/polymeric silicone fluids are linear or cyclic, oligomeric or polymeric silicone (siloxane) fluids. In accordance with the some embodiments, the oligomeric/polymeric silicone fluids increase the flux of drug from the compositions through the skin. While not wanting to be bound by any theory, it is believed that the oligomeric/polymeric silicone fluids impact drug flux by affecting the thermodynamic activity of the drug, such as by affecting the solubility of the drug in the composition In accordance with the some embodiments, the drug-containing polymer matrix comprises one or more primary or secondary amine drugs, such as free base forms thereof.

Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free of” as used herein means that the described composition (e.g., polymer matrix, etc.) comprises less than about 5%, less than about 3%, or less than about 1% by weight, based on the total weight of the composition at issue, of the excluded component(s). The phrase “free of ” as used herein means that the described composition (e.g., polymer matrix, etc.) is formulated without adding the excluded component(s) as an intended component, although trace amounts may be present in other components or as a by-product or contaminant, such that the composition comprises at most only trace amounts of the excluded component(s).

As used herein “subject” denotes any mammal in need of drug therapy, including humans. For example, a subject may be suffering from or at risk of developing a condition that can be treated or prevented with an amine-functional drug, or may be taking an amine-functional drug for other purposes.

As used herein, the terms “topical” and “topically” mean application to a skin or mucosal surface of a mammal, while the terms “transdermal” and “transdermal” connote passage through the skin or mucosa (including oral, buccal, nasal, rectal and vaginal mucosa), into systemic circulation. Thus, the compositions described herein may be applied topically to a subject to achieve transdermal delivery of an amine-functional drug.

As used herein, the phrases “therapeutically effective amount” and “therapeutic level” mean that drug dosage or plasma concentration in a subject, respectively, that provides the specific pharmacological effect for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

The compositions described herein are in a “flexible, finite form.” As used herein, the phrase “flexible, finite form” means a substantially solid form capable of conforming to a surface with which it comes into contact, and capable of maintaining contact so as to facilitate topical application. Such systems in general are known in the art and commercially available, such as transdermal drug delivery patches.

In some embodiments, the compositions described herein comprise a drug-containing polymer matrix that releases drug upon application to the skin (or any other surface noted above). As used herein, “drug-containing polymer matrix” refers to a polymer composition which contains one or more drugs or pharmaceutically acceptable salt thereof and a polymer, such as a pressure-sensitive adhesive polymer or a bioadhesive polymer. A polymer is an “adhesive” or “bioadhesive” if it has the properties of adhesiveness per se. Other polymers can function as an adhesive or bioadhesive by the addition of tackifiers, plasticizers, crosslinking agents or other excipients. Thus, in some embodiments, the polymer matrix optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

In some embodiments, the compositions in flexible, finite form are “monolithic” or “monolayer” systems, such that the drug-containing polymer matrix layer is the only polymeric layer present other than the backing layer and the release liner, if present. In such embodiments, the polymer matrix functions as both the drug carrier and the means of affixing the system to the skin or mucosa. In other embodiments, the compositions in flexible, finite form are multilayer systems, comprising one or more layers in addition to the drug-containing polymer matrix layer, such as one or more additional adhesive layers (such as one or more additional pressure-sensitive adhesive layers), rate-controlling layers (such as one or more rate controlling membranes), and/or other layers useful in a transdermal drug delivery system. In accordance with such embodiments, the drug-containing polymer matrix layer may or may not be a pressure-sensitive adhesive layer and may or may not function to affix the system to the skin. For example, one or more separate adhesive layers may be provided that affixes the system to the skin.

The transdermal drug delivery system also may include a drug impermeable backing layer or film. In some embodiments, the backing layer is adjacent one face of the polymer matrix layer. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak™ 1012 or 9732 backing material (a polyester film with an ethylene vinyl acetate copolymer heat seal layer) is useful in the transdermal drug delivery systems described herein.

The transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include the commercially available products of Dow Corning Corporation designated Bio-Release® liner and Syl-off® 7610 (both silicone-based), Loparex's silicone-coated PET release liner films and 3M's Scotchpak™ 1020, 1022, 9741, 9742, 9744, 9748 and 9755 (fluoropolymer coated polyester films).

The transdermal drug delivery system may be packaged or provided in a package, such as a pouchstock material used in the prior art for transdermal drug delivery systems in general or for transdermal drug delivery systems for the specific drug being formulated. For example, DuPont's Surlyn® can be used in a pouchstock material.

Oligomeric/Polymeric Silicone Fluies

As used herein, the term “oligomeric/polymeric silicone fluids” is used interchangeably with the term “oligomeric/polymeric siloxane fluids.” The oligomeric/polymeric silicone fluids described herein have repeating —Si(CH₃)₂—O— units, and thus have a backbone structure of alternating silicone and oxygen atoms, with hydrocarbon groups attached to the silicone side chain:

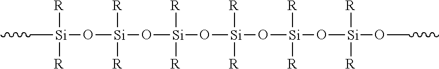

The oligomeric/polymeric silicone fluids described herein exhibit an unusual combination of having both a very strong but very flexible silicon-oxygen inorganic chain (which often is associated with high surface energy) and organic methyl side groups (which often are associated with low surface energy).

Their unique properties include:
A low surface tension, capable of wetting itself and most surfaces, good film formation and surface covering.
High thermal stability, low intermolecular interactions, inert, nonreactive.
High free volume (as compared to analogous hydrocarbons), and so very permeable to various substances and gases.
Good electrical insulating characteristics (dielectric) over a wide range of temperatures and frequencies.
Unique sensory feel and lubricity; non-stinging, biocompatible with skin.
Does not foster the growth of microorganisms.
Insoluble in water (water repellent); good solubility in hydrocarbons.
Low glass transition temperature (Tg: 140 K compared to 200 K for analogous hydrocarbons).
Very low vapor pressure, high flash point.
Clear, colorless, and essentially odorless.

The following table illustrates several oligomeric linear silicone fluids as described herein. The polymeric silicone fluids have a similar chemical structure but more repeating units. Oligomeric/polymeric silicone fluids having 6, 7, 8, 9, 10, 11, 12, 13, 14, 15, or more, repeating units are expressly contemplated. In general, there is no limit on the number of repeating units, although typically the polymers will be designed to be liquid at about 25° C. The sub stituent group on the silicone moieties (shown below as methyl groups, e.g., repeating dimethylsiloxane units) can be selected and controlled depending on the desired properties of the silicone fluid.

| Short Name | L2 | L3 | L4 | L5 |
|---|---|---|---|---|
| Chemical Name | Hexamethyl- | Octamethyl- | Decamethyl- | Dodecamethyl- |
| | disiloxane | trisiloxane | tetrasiloxane | pentasiloxane |
| International | Disiloxane | Trisiloxane | Tetrasiloxane | Dimethicone |
| Nomenclature of | | | | |
| Cosmetic Ingredient | | | | |
| (INCI) | | | | |
| Chemical Structure | 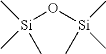 | 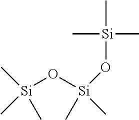 | 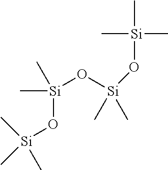 | 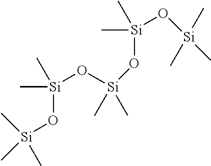 |
| Molecular Weight | 162.38 | 236.53 | 310.68 | 384.84 |
| Melting Point | −59 | −82 | −90 | −81 |
| (° C.) | | | | |
| Boiling Point | 100 | 151 | 194 | 229 |
| (° C.) | | | | |
| Density | 0.76 | 0.82 | 0.85 | 0.87 |
| (g/cm³ at RT) | | | | |
| Vapor Pressure | 5626.1 | 519.9 | 57.3 | 13.6 |
| (Pa at RT) | | | | |
| Water Solubility | ~0 | ~0 | ~0 | ~0 |
| (mg/L at RT) | | | | |
| Octanol/Water | 4.2 | 4.8 | 5.4 | 6.0 |
| Partition Coefficient | | | | |
| (Log K_{ow} at RT), | | | | |

The following table illustrates several oligomeric cyclic silicone fluids as described herein. The substituent group on the silicone moieties (shown below as methyl groups, e.g., with unmodified dimethyl silicone units in a cyclical structure, e.g., cyclomethicone) can be selected and controlled depending on the desired properties of the silicone fluid. In some embodiments, the cyclic silicone fluids are “oligomers” with 3-6 silicone atoms, as illustrated below. Cyclic silicone fluids having more silicone atoms, such as 7, 8, 9, 10, or more are expressly contemplated. As noted above, the polymers typically will be designed to have a melting point below 25° C. so that the polymer is a liquid at 25° C.

| Short Name | D3 | D4 | D5 | D6 |
|---|---|---|---|---|
| Chemical Name | Hexamethyl- | Octamethyl- | Decamethyl- | Dodecamethyl- |
| | cyclotrisiloxane | cyclotetrasiloxane | cyclopentasiloxane | cyclohexasiloxane |
| International | cyclotrisiloxane | cyclotetrasiloxane | cyclopentasiloxane | cyclohexasiloxane |
| Nomenclature | | | | |
| of Cosmetic Ingredient | | | | |
| (INCI) | | | | |
| Chemical Structure | 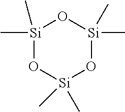 | 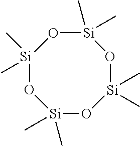 | 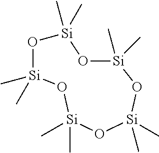 | 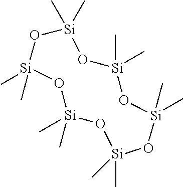 |
| Molecular Weight | 222.47 | 296.62 | 370.77 | 444.93 |
| Melting Point | 64 | 18 | −47 | −3 |
| (° C.) | | | | |
| Boiling Point | 134 | 176 | 210 | 245 |
| (° C.) | | | | |
| Density | | 0.950 | 0.958 | 0.963 |
| (g/cm³ at RT) | | | | |
| Vapor Pressure | 470.6 | 140.0 | 33.2 | 6.6 |
| (Pa at RT) | | | | |
| Water Solubility | ~0 | ~0 | 0.017-0.050 | 0.005 |
| (mg/L at RT) | | | | |
| Octanol/Water | 4.5 | 5.1 | 5.2-5.7 | 6.3 |
| Partition Coefficient | | | | |
| (Log K_{ow} at RT), | | | | |

In some embodiments, a drug-containing polymer matrix is provided that comprises one or more oligomeric/polymeric silicone fluids as described herein. In specific embodiments, the drug-containing polymer matrix comprises (i) a polymer matrix, such as a pressure-sensitive adhesive polymer matrix, (ii) one or more oligomeric/polymeric silicone fluids as described herein, and (iii) one or more drugs.

In some embodiments, formulating the drug-containing polymer matrix with the one or more oligomeric/polymeric silicone fluids achieves greater drug flux (e.g., enhanced permeation through the skin) than is achieved with a comparable polymer matrix that does not include the one or more oligomeric/polymeric silicone fluids. As noted above, while not wanting to be bound by any theory, it is believed that the oligomeric/polymeric silicone fluids impact drug flux by affecting the thermodynamic activity of the drug, such as by affecting the solubility of the drug in the composition.

In some embodiments, formulating the drug-containing polymer matrix with the one or more oligomeric/polymeric silicone fluids permits higher or lower drug loading than could be achieved with a comparable polymer matrix that does not include the one or more oligomeric/polymeric silicone fluids. In specific embodiments, a drug-containing polymer matrix as described herein comprises one or more drugs at or near its saturation concentration in the polymer matrix. Thus, in some embodiments, an oligomeric/polymeric silicone fluid as described herein is included in a trandsermal drug delivery system as a solubility modifier. For example, an oligomeric/polymeric silicone fluid as described herein may be included in a drug-containing polymer matrix to modify (e.g., increase or decrease) the solubility of one or more drugs in the polymer matrix.

In some embodiments, a transdermal drug delivery system is provided that comprises a drug-containing polymer matrix that comprises one or more oligomeric/polymeric silicone fluids as described herein. In specific embodiments, the transdermal drug delivery system and achieves a desired pharmacokinetic profile, such as a therapeutically effective permeation rate of drug into and through the skin for therapeutic effect.

In some embodiments, the drug-containing polymer matrix as described herein exhibits acceptable chemical stability with regard to the drug and/or other components of the polymer matrix.

In some embodiments, the drug-containing polymer matrix exhibits acceptable physical properties, such as acceptable shear, tack, and/or peel properties, and/or acceptable wear properties.

Polymer Matrix

The other components of the polymer matrix are not limited, but can include any that are used in transdermal drug delivery systems.

The carrier polymer of the polymer matrix may be any polymer suitable for use in a transdermal drug delivery system. For example, the carrier polymer may be a hydrophilic polymer approved for pharmaceutical use such as an acrylic polymer, rubber polymer, cellulose polymer, or mixture thereof In some embodiments, the carrier polymer is a pressure-sensitive adhesive, such as an acrylic pressure-sensitive adhesive, or rubber-based pressure-sensitive adhesive such as those exemplified below. In specific embodiments, the carrier polymer is chemically compatible with the one or more drugs being formulated. For example, when the drug comprises methylphenidate, the carrier polymer may be one that does not include functional groups that are reactive with methylphenidate, such as one or more non-functional acrylic polymers. Similarly, when the drug comprises amphetamine, the carrier polymer may be one that does not include functional groups that are reactive with amphetamine, such as one or more non-acid functional acrylic polymers.

In some embodiments, the polymer matrix does not include any silicone polymers other than the oligomeric/polymeric silicone fluids described herein. Thus, in some embodiments, the polymer matrix is substantially free of, or is free of, polymers other than the oligomeric/polymeric silicone fluids described herein. For example, in some embodiments, the polymer matrix is substantially free of, or is free of, silicone pressure-sensitive adhesives, including amine-compatible and/or end-capped silicone pressure-sensitive adhesives.

Acrylic Polymers

In some embodiments, the polymer carrier comprises an acrylic polymer. The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the acrylic polymer constitutes from about 2% to about 95% by weight of the polymer content of the polymer matrix, including about 3% to about 90% and about 5% to about 85%, such as 2% to 95%, 3% to 90% and 5% to 85%. In some embodiments, the amount and type of acrylic polymer is dependent on the type and amount of drug being formulated used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, methyl methacrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters. Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold by Henkel North America under the Duro-Tak® trade name (such as Duro-Tak® 87-2287, -4098, -2852, -2196, -2296, -2194, -2825, -2516, -2070, -2353, -2154, -2510, -4852, -9085, -9088, -9900, -2051, -2052, -2054, 235A, -2074, -2979, -2525, -2677, -4287, -502A, -503A, -504A, -900A, -901A and -9301) and under the GELVA® GMS trade name (such as GELVA® GMS 2480, 788, 7883, 737, 263, 1430, 1753, 1151, 2450, 2495, 2499, 3067, 3071, 3083, 3087, 3235, 9073 and 9083). Other suitable acrylic adhesives include those sold under the trademark EUDRAGIT® by Evonik Industries AG, Essen, Germany.

Rubber Polymers

As noted above, in some embodiments the polymer matrix comprises one or more rubber-based polymers, such as one or more rubber-based pressure-sensitive adhesives, such as natural or synthetic polyisoprene, polybutylene, polyisobutylene, styrene-butadiene polymers, styrene-isoprene-styrene block copolymers (such as Kraton® D111 KT), hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. Additionally or alternatively, as discussed above, the polymer matrix may comprise a non-adhesive polymer, such as ethyl cellulose.

Other Components

In accordance with any of the embodiments described herein, the drug-containing polymer matrix may comprise one or more other pharmaceutically acceptable excipients, such as a plasticizer, penetration enhancer, filler, and the like. In some embodiments, the polymer matrix comprises from about 0% to about 20% of one or more such excipients.

A “penetration enhancer” is an agent known to accelerate the delivery of the drug through the skin by changing the permeation of the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In some embodiments, the polymer matrix or transdermal drug delivery system does not include a penetration enhancer.

The polymer matrix and/or face adhesive may further comprise various thickeners, fillers, and other additives or components known for use in transdermal drug delivery systems to further modify properties of the matrix or face adhesive, such as polyvinylpyrrolidone (PVP), ethylene-vinyl acetate copolymers, cellulose derivatives, SiO₂, and other components.

In accordance with any of the embodiments described herein, the polymer matrix may comprise an antioxidant. In specific embodiments the antioxidant may be one known for use in transdermal drug delivery systems, such as butylhydroxytoluene (BHT), butylhydroxyanisole (BHA), tertiary-butyl hydroquinone (TBHQ), ascorbic acid, ascorbyl palmitate, alpha-tocopherol and its esters, fumaric acid, malic acid, sodium ascorbate, sodium metabisulfite, and propyl gallate, and mixtures thereof. The antioxidant may comprise from about 0 to about 1%, including from about 0 to about 0.5% by weight of the polymer matrix.

Drugs

The oligomeric/polymeric silicone fluids described herein are useful in transdermal drug delivery systems for formulating any drug that can delivered transdermally. In some embodiments, the one or more drugs comprises one or more amine-functional drugs. The term “amine-functional” denotes a drug or active agent that contains one or more primary amine radicals such as phenylpropanolamine, secondary amine radicals such as propranolol, tertiary amine radicals such as theophylline and chlorpheniramine. The term “amine-functional” also includes heterocyclic amine radicals such as those found in theophylline and diethylcarbomazine and salts of amine-functional drugs such as scopolamine hydrobromide provided that they can be delivered transdermally, but does not include oxidized nitrogen radicals such as nitro radicals. Other examples of amine-functional drugs for transdermal drug delivery include, for example, tetracain, ephedrine, clonidine, nicotine, ramipril, enalapril, fentanyl and analogs such as alfentanyl, carfentanyl, lofentanyl, remifentanyl, sufentanyl, and trefentanyl, amphetamine, dextroamphetamine, methamphetamine, and atropine. Further examples of amine-functional drugs for use in transdermal drug delivery systems will be apparent to those skilled in the art. In specific embodiments, the one or more drugs comprises one or more of amphetamine, methylphenidate, rivastigmine or clonidine.

In specific embodiments, the one or more drugs comprises one or more primary or secondary amine free base drugs. Such drugs tend to interact with silicone resin that is present in the silicone pressure-sensitive adhesives, resulting in drug instability and peel problems (e.g., difficulty peeling the matrix off the release liner). It was surprisingly and unexpectedly found that by formulating such drugs in polymer matrices as described herein, comprising one or more oligomeric/polymeric silicone fluids as described herein, these problems are reduced, minimized, or avoided.

The amount of drug formulated in a drug-containing polymer matrix as described herein will vary with the specific drug being formulated, the specific polymer matrix, and the specific desired pharmacokinetic profile and/or thereapeutic effect. In general the amount of drug will be up to about 30% by weight of the drug-containing polymer matrix.

The drug-containing polymer matrix may comprise drug in its free base or free acid form, or as any pharmaceutically acceptable salt or ester thereof, or any combinations thereof. Exemplary suitable pharmaceutically acceptable salts are salts of weak inorganic and organic acids, and quaternary ammonium salts. These include without limitation, salts with acids such as sulfuric, phosphoric, hydrochloric, hydrobromic, hydriodic, sulfamic, citric, lactic, maleic, malic, succinic, tartaric, cinnamic, acetic, benzoic, gluconic, or ascorbic acid, or quaternary ammonium salts with organic esters of sulfuric, hydrohalic, or aromatic sulfonic acids, such as methyl chloride, methyl bromide, ethyl chloride, propyl chloride, butyl chloride, isobutyl chloride, benzylchloride, benzyl bromide, phenethyl bromide, naphthymethyl chloride, dimethyl sulfate, methyl benzenesulfonate, ethyl toluenesulfonate, ethylene chlorohydrin, propylene chlorobydrin, allyl bromide, methylallyl bromide or crotyl bromide esters.

In some embodiments, a drug-containing matrix as described herein comprises a drug, an acrylic pressure-sensitive adhesive polymer, an oligomeric/polymeric silicone fluid as described herein, and optionally, additional excipients as desired.

As noted above, in some embodiments, the drug-containing polymer matrix comprises up to about 10%, up to about 20%, or up to about 30% by weight drug, such as 0.1 to 10% or 0.1 to 20% or 0.1 to 30% by weight drug.

In some embodiments, the drug-containing polymer matrix comprises from about 2.5% to about 50% by weight oligomeric/polymeric silicone fluid, such as about 2.5%, 5%, 7.5%, 10%, 12.5%, 15%, 20%, 30%, 40%, or 50% by weight oligomeric/polymeric silicone fluid. In some embodiments, the drug-containing polymer matrix comprises about 20% by weight oligomeric/polymeric silicone fluid.

In some embodiments, the drug-containing polymer matrix comprises up to about 95% by weight carrier polymer, such as from about 5 to 95% by weight carrier polymer, including about 50%, 55%, 60, 65%, 70% 75%, 80%, 85%, and 90% carrier polymer. As used herein, the term “carrier polymer” includes blends and mixtures of two or more carrier polymers, such as any two or more carrier polymers and pressure-sensitive adhesive polymers discussed herein.

In some embodiments, the drug-containing polymer matrix comprises up to about 20% by weight of one or more excipients.

Transdermal Drug Delivery Systems

As noted above, described herein are transdermal drug delivery systems that comprise a polymer matrix that comprises one or more oligomeric/polymeric silicone fluids as described herein. As noted above, the carrier polymer may or may not be a pressure sensitive adhesive. Further, as noted above, the transdermal drug delivery system may be a monolithic device comprised of the polymer matrix, or may include one or more additional layers, such as a face adhesive layer, or may be provided with a surrounding adhesive portion. As noted above, the transdermal drug delivery system may include a backing layer on one side of the polymer matrix layer and a release liner on the other side of the polymer matrix layer. In multilayer systems, the polymer matrix layer may be the skin-contacting layer (e.g., directly adjacent the release liner) or may be separated from the skin by one or more intervening layers, and may or may not be directly adjacent the backing layer. Further, an optional overlay adhesive film may be used to strengthen the adhesion of the patch to the skin.

In some embodiments, the system consists essentially of the polymer matrix layer. By “consists essentially of the polymer matrix layer” means that the system does not contain any other layers that affect drug delivery, such as an additional rate-controlling polymer layer, rate-controlling membrane, or drug reservoir layer. It will be understood, however, that the system that consists essentially of the polymer matrix layer may comprise a backing layer and/or release liner.

The system may be of any shape or size suitable for transdermal application and of an appropriate sizes for application to deliver the desired dose, such as ranging from about 2 cm² to about 50 cm², including about 5 cm², about 10 cm², about 20 cm², about 25 cm², about 30 cm², about 35 cm², about 40 cm², about 45 cm², about 50 cm², about 60 cm², and about 75 cm².

In specific embodiments relating to amphetamine, amphetamine can be present in an amount from about 0.5 mg/cm² to about 3 mg/cm², based on the active surface area of the of the polymer matrix (e.g., the surface area of the drug-containing polymer matrix), such as about 1 mg/cm², including about 1.05 mg/cm², based on the active surface area of the of the polymer matrix. Other exemplary amounts include about 0.75 mg/cm², 0.8 mg/cm², 0.9 mg/cm², 1.0 mg/cm², 1.05 mg/cm², 1.1 mg/cm², 1.2 mg/cm², and 1.25 mg/cm², 1.5 mg/cm², 2.0 mg/cm², 2.5 mg/cm², and 3.0 mg/cm². In accordance with any of thee embodiments described herein, the system may include from about 5 to about 30 mg of amphetamine base or an equivalent amount of a pharmaceutically acceptable salt or prodrug thereof, including about 5, 10, 15, 20, 25, or 30 mg of amphetamine base or equivalent.

In specific embodiments relating to rivastigmine, the drug-containing polymer matrix has a size of about 16-24 cm², such as 17.5 cm², 18 cm², 19 cm², or 23.5 cm² and contains about 60-65 mg rivastigmine per unit dose (including 61.9 mg, 63.9 mg, 64 mg) and/or delivers a dose of about 4.6 mg/day. In other specific embodiments, the system is about 32-48 cm², such as 35 cm², 36 cm², 38 cm², or 47 cm², and contains about 126 mg rivastigmine per unit dose and/or delivers a dose of about 9.5 mg/day. In further specific embodiments relating to rivastigmine, the system contains about 32-65 mg rivastigmine per unit dose, or about 67-126 mg rivastigmine per unit dose.

In specific embodiments relating to methylphenidate, the methylphenidate can be present in an amount from about 0.5 mg/cm² to about 5 mg/cm², based on the active surface area of the of the polymer matrix, including from about 1.2 mg/cm² to about 3 mg/cm²,. Exemplary amounts include about 0.5 mg/cm², about 0.8 mg/cm², about 1 mg/cm², about 1.2 mg/cm², about 1.4 mg/cm², about 1.6 mg/cm², about 1.7 mg/cm², about 1.8 mg/cm², about 2.0 mg/cm², about 2.2 mg/cm², about 2.4 mg/cm², about 2.6 mg/cm², about 2.8 mg/cm², about 3.0 mg/cm², about 3.3 mg/cm², about 3.5 mg/cm², about 3.7 mg/cm², about 3.9 mg/cm², about 4.1 mg/cm², about 4.3 mg/cm², about 4.5 mg/cm², about 4.7 mg/cm², and about 5.0 mg/cm². In accordance with any of the methylphenidate embodiments, the size of the drug-containing polymer matrix can be, for example, in the range of from about 2 cm² to about 60 cm², including from about 15 cm² to about 30 cm², such as about 6 cm ², 8 cm², 10 cm², 12.5 cm², 14.5 cm², 15 cm², 18.75 cm², 22.5 cm², 25 cm², 27.5 cm², 30 cm², 37.5 cm², and 45 cm². In accordance with any of the methylphenidate embodiments described herein, the polymer matrix may include from about 20 to about 225 mg methylphenidate base or an equivalent amount of a pharmaceutically acceptable salt thereof.

In specific embodiments relating to clonidine, the clonidine may be present in the polymer matrix at an amount from about 0.1% to about 50%, including from about 1% to about 20%, such as from about 1% to about 10% by weight, such as about 1, about 2, about 3, about 4, about 5, about 6, about 7, about 8, about 9 or about 10% by weight, based on the total dry weight of the polymer matrix. In specific embodiments, the polymer matrix comprises about 3, about 4, about 5, about 6, or about 7% by weight clonidine, based on the total dry weight of the polymer matrix. In accordance with any of the clonidine embodiments, the composition may be designed to deliver from about 0.05 to about 0.5 mg clonidine per day, such as about 0.05, 0.1, 0.2, 0.3, 0.4 or 0.5 mg clonidine per day, and may be designed for use over a period of time from 1 to about 7 days, or longer. In accordance with any of the clonidine embodiments, the size of the drug-containing polymer matrix can be, for example, in the range of from about 2 cm² to about 60 cm², including from about 2 cm² to about 15 cm², such as about 2 cm², 3.5 cm², 7 cm², 10.5, or 12 cm².

The polymer matrices described herein may be prepared by methods known in the art. For example, a polymer matrix can be prepared by blending the components of the polymer matrix, applying the matrix material to a support layer such as a backing layer or release liner (such as by calender coating, hot melt coating, solution coating, etc.), and removing any remaining solvents. The polymer matrices can be formed into systems by methods known in the art, such as by die-cutting into sizes and shapes suitable for use.

The amine drug can be added at any stage. In one embodiment, all polymer matrix components, including the drug, are blended together. The order of steps, amount of ingredients, and the amount and time of agitation or mixing can be determined and optimized by the skilled practitioner. An exemplary general method is as follows:

Appropriate amounts of solvent(s), enhancer(s), and organic solvent(s) (for example toluene, or ethyl acetate and/or isopropyl alcohol) are combined and thoroughly mixed together in a vessel.

The drug and, optionally, antioxidant, are added to the mixture and agitation is carried out until the drug is uniformly mixed in.

Appropriate amounts of polymer components, oligomeric/polymeric silicone fluid, and other excipients are then added to the drug mixture, and thoroughly mixed.

The formulation is then transferred to a coating operation where it is coated onto a protective release liner at a controlled specified thickness. The coated product is then passed through an oven in order to drive off all volatile processing solvents.

The dried product on the release liner is then joined to the backing material and wound into rolls for storage.

Appropriate size and shape “systems” are die-cut from the roll material and then pouched.

Other manufacturing methods are known in the art that are suitable for making the systems described herein.

In some embodiments, the coat weight of the polymer matrix is selected and tailored to control and/or optimize the drug delivery profile. For example, systems with a higher coat weight (e.g., unit weight of polymer matrix per unit area of system) may achieve increased drug flux and improved flux profile.

Therapeutic Methods

In some embodiments, there is provided a method of effecting transdermal drug delivery of a drug, by applying a system as described herein to the skin or mucosa of a subject in need thereof. In some embodiments, the system is applied over a period of at least about 1 day, at least about 2 days, at least about 3 days, at least about 4 days, at least about 5 days, at least about 6 days, or at least about 7 days, such as for 1, 2, 3, 4, 5, 6 or 7 days, or longer. In some embodiments, the method is effective to achieve transdermal delivery of therapeutically effective amounts of drug during the application period. In some embodiments, the method is effective to achieve therapeutic levels of drug in the subject during the application period. In some embodiments, the method is effective to achieve a substantially constant rate of drug delivery over a period of at least about 1 day, at least about 2 days, at least about 3 days, at least about 4 days, at least about 5 days, at least about 6 days, or at least about 7 days, or longer.

In some embodiments, the amphetamine systems described herein are used for stimulating the central nervous system, treating attention deficit disorder (ADD), or treating narcolepsy.

In some embodiments, the rivastigmine systems described herein are designed for use by patients suffering from or at risk of developing dementia associated with Alzheimer's disease or Parkinson's disease.

In some embodiments, the methylphenidate systems described herein are designed for use by patients suffering from attention deficit disorder (ADD) or attention deficit hyperactivity disorder (ADHD), postural orthostatic tachycardia syndrome, or narcolepsy.

In some embodiments, the clonidine systems described herein are used for treating hypertension (high blood pressure), attention deficit disorder (ADD), attention deficit hyperactivity disorder (ADHD), anxiety disorders, withdrawal (e.g. from alcohol, opioids or nicotine), migraine, menopausal flushing, diarrhea, or pain.

The following specific examples are included as illustrative of the transdermal drug delivery systems and polymer matrices described herein. These examples are in no way intended to limit the scope of the invention. Other aspects of the invention will be apparent to those skilled in the art to which the invention pertains.

### EXAMPLE 1

### Miscibility of Silicone Fluids in Acrylic Pressure-Sensitive Adhesives

Compositions comprising an acrylic pressure-sensitive adhesive and oligomeric/polymeric silicone fluid as described herein were prepared as follows and applied to a release liner, and assessed by visual observation after 1 week. Satisfactory performances was indicated by an absence of phase separation/oil droplets, uniform/even coating, and/or normal peel from the release liner.

| | Dimethicone | Dimethicone | ST-Cyclomethicone | Simethicone |
|---|---|---|---|---|
| Acrylic PSA | (Q7-9120, 20 CST) | (Q7-9120, 12500 CST) | (5-NF) | (Q7-2243LVA) |
| GMS 3087 | 7.5% | 2.5% | 12.5% | 2.5% |
| (non-functional) | | | | |
| GMS 788 | 20% | 2.5% | 40% | 7.5% |
| (hydroxyl-functional | | | | |
| Duro-Tak 87-2194 | 20% | 7.5% | 30% | 7.5% |
| (carboxy-functional, | | | | |
| cross-linked) | | | | |
| Duro-Tak 87-2852 | 7.5% | 7.5% | 50% | 7.5% |
| (carboxy-functional, | | | | |
| non cross-linked) | | | | |

TABLE 2
Adhesive laminate Physical Testing (T = 1 week)

| | | | | |
|---|---|---|---|---|
| adhesive | Dimethicone (low | Dimethicone | | |
| only | viscosity: 20 CST) | (high viscosity: 12500 CST) | ST-Cyclomethicone | Simethicone |

Probe Tack g (Mean ± SD, n = 5)

| | | | | | |
|---|---|---|---|---|---|
| GMS 3087 | 632 ± 91 | 7.5%: 259 ± 73 | 2.5%: 493 ± 62 | 12.5%: 550 ± 98 | 2.5%: 556 ± 92 |
| GMS 788 | 657 ± 61 | 20%: 250 ± 20 | 2.5%: 600 ± 51 | 40%: 419 ± 53 | 7.5%: 478 ± 43 |
| DT 87-2194 | 632 ± 119 | 20%: 370 ± 40 | 7.5%: 568 ± 45 | 30%: 639 ± 109 | 7.5%: 633 ± 64 |
| DT 87-2852 | 326 ± 31 | 7.5%: 251 ± 68 | 7.5%: 425 ± 34 | 50%: 101 ± 43 | 7.5%: 370 ± 82 |

Shear at 500 g, min (Mean ± SD, n = 3)

| | | | | | |
|---|---|---|---|---|---|
| GMS 3087 | 77 ± 12 | 7.5%: 12 ± 4 | 2.5%: 33 ± 9 | 12.5%: 16 ± 3 | 2.5%: 28 ± 4 |
| GMS 788 | 41 ± 3 | 20%: 6 ± 1 | 2.5%: 25 ± 5 | 40%: 5 ± 0 | 7.5%: 20 ± 4 |
| DT 87-2194 | >1200 | 20%: adhesion failure | 7.5%: adhesion failure | 30%: 725 ± 168 (n = 2) | 7.5%: 159 ± 95 (n = 2) |
| DT 87-2852 | >1200 | 7.5%: >1100 | 7.5%: 392 ± 274 | 50%: adhesion failure | 7.5%: >1100 |

### EXAMPLE 2

### Skin Permeation Study with Amphetamine

Amphetamine compositions were prepared as described below, and drug flux (μg/cm²/hr) through human cadaver skin was assessed.

| | | Peel from | Shear at | Tack | Peel off from the |
|---|---|---|---|---|---|
| | | stainless steel (g) | 500 g (min) | (g) | liner |
| Amphetamine | 67.5% GMS 3087 | 144 ± 12 | 46 ± 7 | 127 ± 48 | easy |
| | 17.5% Duro-Tak 900A | | | | (after 4 months) |
| | 15% Amphetamine | | | | |
| Amphetamine + | 51.6% GMS 3087 | 79 ± 16 | 13 ± 1 | 163 ± 71 | easy |
| 20% | 13.4% Duro-Tak 900A | | | | (after 4 months) |
| Cyclomethicone | 15% Amphetamine | | | | |
| | 20% Cyclomethicone | | | | |

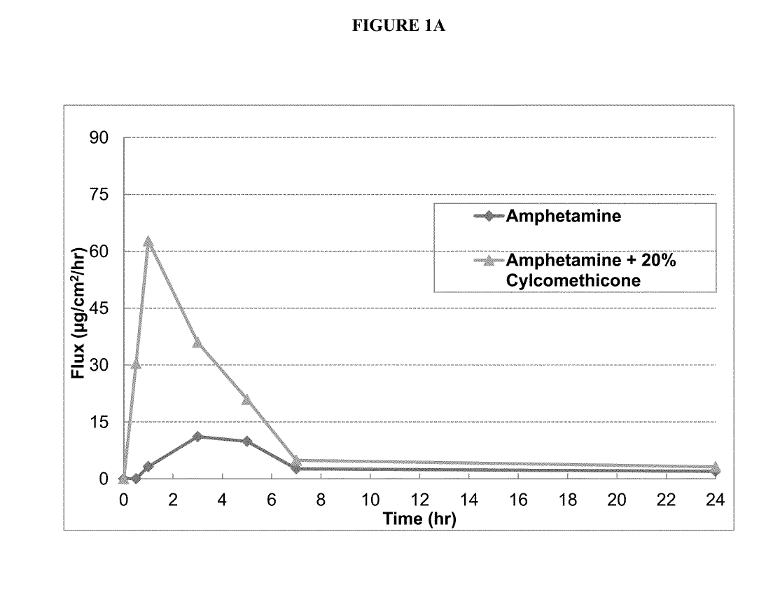

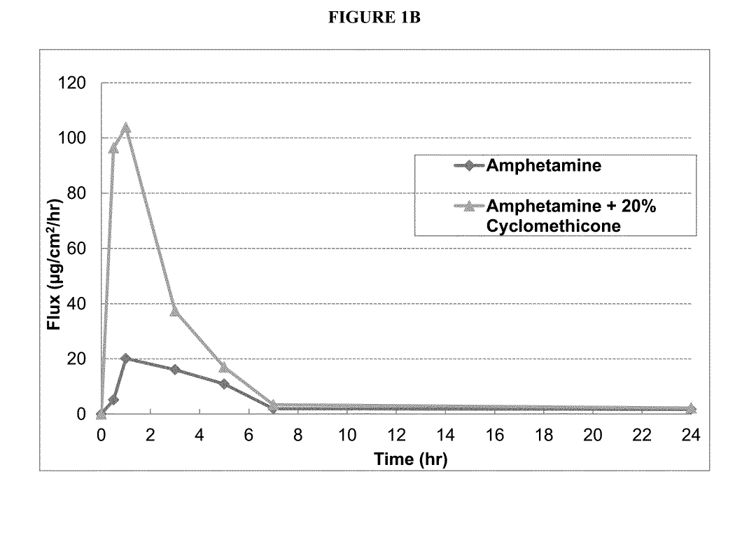

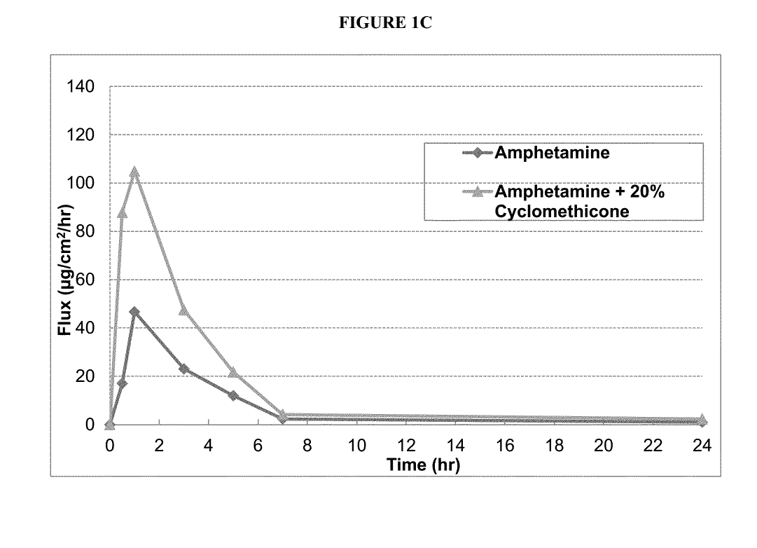

Results are shown in FIGS. 1A-C. As shown in the figures, the compositions comprising a oligomeric/polymeric silicone fluid as described herein exhibited increased permeation (up to about 5×).

### EXAMPLE 3

### Skin Permeation Study with Rivastigmine

Rivastigmine compositions were prepared as described below, and drug flux (μg/cm²/hr) through human cadaver skin was assessed.

| | | Peel from | Shear at | Tack | Peel off from the |
|---|---|---|---|---|---|
| | | stainless steel (g) | 500 g (min) | (g) | liner |
| Rivastigmine | 80% Duro-Tak 87-2194 | 457 ± 67 | 855 ± 105 | 571 ± 60 | easy |
| | 20% Rivastigmine | | | | (after 4 months) |
| Rivastigmine + | 60% Duro-Tak 87-2194 | 319 ± 57 | 213 ± 62 | 413 ± 83 | easy |
| 20% | 20% Rivastigmine | | | | (after 4 months) |
| Cyclomethicone | 20% Cyclomethicone | | | | |

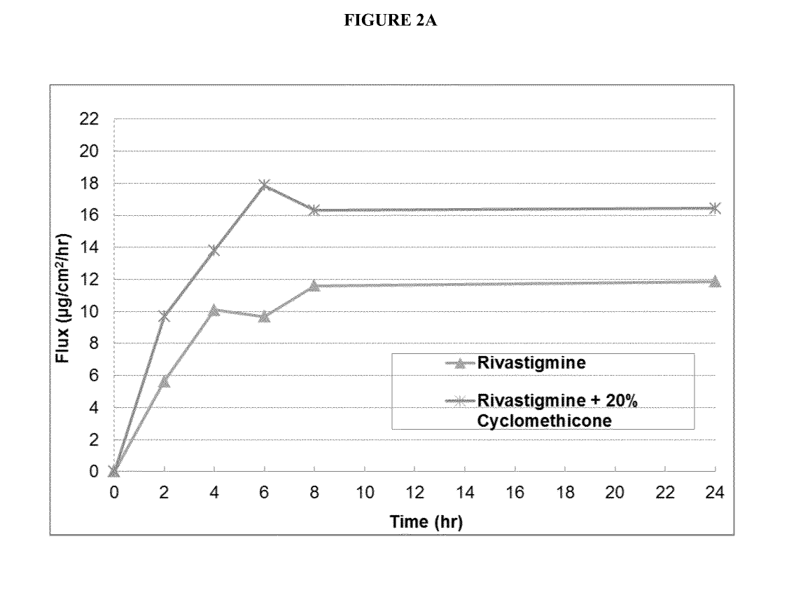

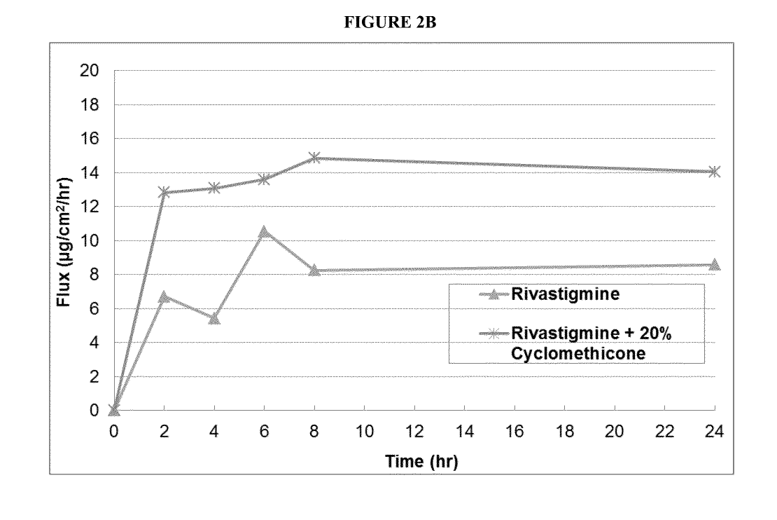

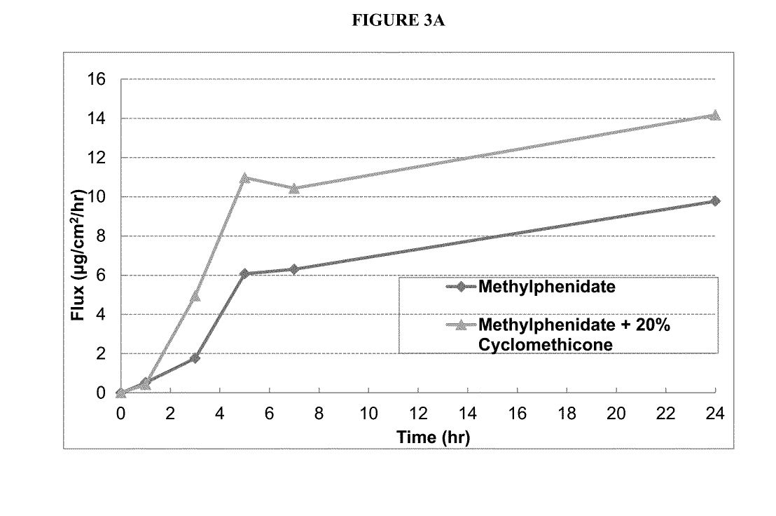

Results are shown in FIGS. 2A-B. As shown in the figures, the compositions comprising a oligomeric/polymeric silicone fluid as described herein exhibited increased permeation (up to about 1.75×).

### EXAMPLE 4

### Skin Permeation Study with Methylphenidate

Methylphenidate compositions were prepared as described below, and drug flux (μg/cm²/hr) through human cadaver skin was assessed.

| | | Peel from | Shear at | Tack | Peel off from the |
|---|---|---|---|---|---|
| | | stainless steel (g) | 500 g (min) | (g) | liner |
| Methylphenidate | 60% GMS 3235 | 458 ± 98 | 8 ± 2 | 244 ± 68 | easy |
| | 15% GMS 3087 | | | | (after 4 months) |
| | 25% Methylphenidate | | | | |
| Methylphenidate + | 44% GMS 3235 | 263 ± 57 | 1 ± 0 | 251 ± 65 | easy |
| 20% | 11% GMS 3087 | | | | (after 4 months) |
| Cyclomethicone | 25% Methylphenidate | | | | |
| | 20% Cyclomethicone | | | | |

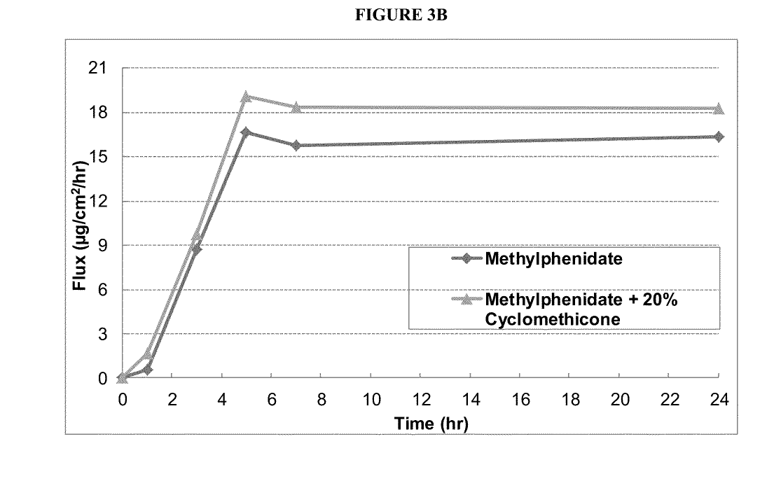

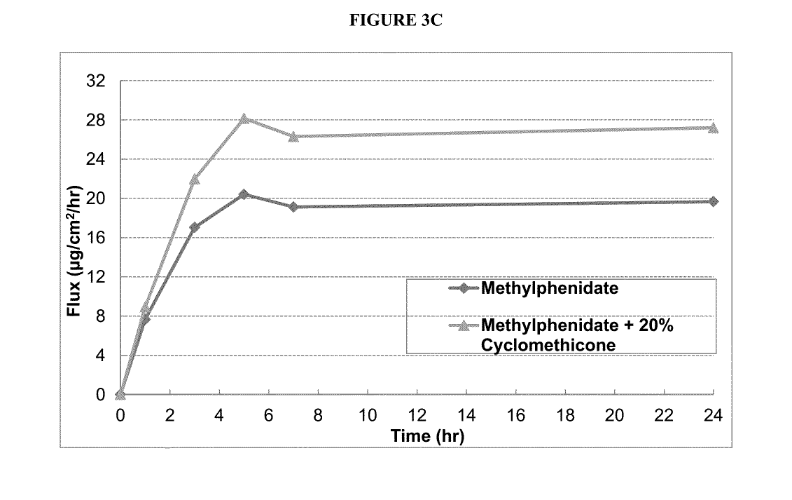

Results are shown in FIGS. 3A-C. As shown in the figures, the compositions comprising a oligomeric/polymeric silicone fluid as described herein exhibited increased permeation (up to about 1.4×).

What is claimed is:

1. A composition for the transdermal delivery of a drug in the form of a flexible finite system for topical application, comprising a drug-containing polymer matrix comprising (i) a drug; (ii) a carrier polymer, and (iii) from 2.5 to 50% by weight of an oligomeric/polymeric silicone fluid having repeating —Si(CH₃)₂—O— units.

2. The composition of claim 1, wherein the oligomeric/polymeric silicone fluid comprises a linear oligomeric/polymeric silicone.

3. The composition of claim 2, wherein the linear oligomeric/polymeric silicone is selected from the group consisting of hexamethyldisiloxane, octamethyltrisiloxane, decamethyltetrasiloxane, and dodecamethylpentasiloxane.

4. The composition of claim 1, wherein the oligomeric/polymeric silicone fluid comprises a cyclic oligomeric/polymeric silicone.

5. The composition of claim 4, wherein the cyclic oligomeric/polymeric silicone fluid is selected from the group consisting of hexamethylcyclotrisiloxane, octamethylcyclotetrasiloxane, decamethylcyclopentaasiloxane, and dodecamethylcyclohexasiloxane.

6. The composition of claim 1, comprising from 7.5 to 20% by weight of the oligomeric/polymeric silicone fluid.

7. The composition of claim 1, wherein the carrier polymer comprises an acrylic polymer.

8. The composition of claim 7, wherein the acrylic polymer is selected from the group consisting of non-functional acrylic polymers, hydroxy-functional acrylic polymers, and carboxy-functional acrylic polymers.

9. The composition of claim 1, wherein the drug is selected from the group consisting of primary and secondary amine drugs in free base form.

10. The composition of claim 1, wherein the drug is selected from the group consisting of amphetamine, rivastigmine, methylphenidate and clonidine.

11. The composition of claim 1, wherein the drug-containing polymer matrix is substantially free of silicone-containing pressure-sensitive adhesives.

12. The composition of claim 1, further comprising a backing layer.

13. The composition of claim 1, further comprising a release liner.

14. A method for the transdermal delivery of a drug, comprising topically applying a composition as claimed in claim 1, to the skin or mucosa of a subject in need thereof.

15. A composition as claimed in claim 1, for use in transdermally delivering a drug.

16. A method of preparing a composition for the transdermal delivery of a drug in the form of a flexible finite system for topical application, comprising preparing a drug-containing polymer matrix comprising (i) a drug; (ii) a carrier polymer, and (iii) from 2.5 to 50% by weight of an oligomeric/polymeric silicone fluid having repeating —Si(CH₃)₂—O— units.

17. The method of claim 16, wherein the drug-containing polymer matrix is prepared without silicone-containing pressure-sensitive adhesives.
